(19) United States
(12) Patent Application Publication
(10) Pub. No.: US 2025/0135402 A1
(43) Pub. Date: May 1, 2025

(54) AIR PURIFICATION SYSTEM

(71) Applicants: Kenji TANIGUCHI, Tottori-City (JP); Kaiser IP Corp., Wetaskiwin (CA)

(72) Inventors: Benjamin KAISER, Wetaskiwin (CA); Kenji TANIGUCHI, Tottori-City (JP)

(21) Appl. No.: 18/932,054

(22) Filed: Oct. 30, 2024

Related U.S. Application Data
(60) Provisional application No. 63/546,337, filed on Oct. 30, 2023.

(51) Int. Cl. B01D 53/82; A61L 9/015; A61L 9/20; A61L 101/02; B01D 53/00; B01D 53/88

(52) U.S. Cl. CPC B01D 53/82; A61L 9/015; A61L 9/205; B01D 53/007; B01D 53/885; A61L 2101/02; A61L 2209/12; B01D 2251/402; B01D 2251/404; B01D 2251/602; B01D 2251/604; B01D 2255/20707; B01D 2255/702; B01D 2257/90; B01D 2257/91; B01D 2258/06; B01D 2259/4508; B01D 2259/804

(57) ABSTRACT

An air purification system is provided that includes a housing and one or more anti-viral units which promote hydroxyl generation in air streams. The housing is configured to allow airflow to travel therethrough and anti-viral unit(s) include a substrate material, and an anti-viral agent material supported on the substrate material. The anti-viral agent material can be a mineral powder containing a metal oxide and a metal hydroxide suitable for hydroxyl generation. The system may also contain at least one light source, and the one or more anti-viral units can also contain activated charcoal and/or titanium dioxide. The air purification system is used in a structure. The system emits hydroxyls into the structure to neutralize viruses, odors, bacteria, and/or other pathogens in the structure.

### CROSS-REFERENCE TO RELATED APPLICATION

This application claims priority of U.S. Provisional Patent Application Ser. No. 63/546,337, entitled Air Purification System, filed Oct. 30, 2023, and hereby incorporates this provisional patent application by reference herein in its entirety.

### FIELD OF TECHNOLOGY

The following relates to farm air purification systems. More particularly, the following relates to air purification systems comprising one or more anti-viral units including dolomite minerals to promote hydroxyl generation in air streams.

### BACKGROUND

The Avian Influenza is a highly contagious viral infection impacting the agricultural industry. Treating barn air to reduce or eliminate pathogens is challenging due to multiple points of entry to these structures, biosecurity issues with people, biosecurity issues with equipment, pest invasion including mice, rats, and insects, air velocity, and high air exchange rates.

Air purification systems exist for commercial buildings to treat the air space for viruses and other pathogens as the air is recirculated multiple times per hour. However, these commercial air purification systems may not be a suitable option for purifying air within agricultural structures. In barns and other similar agricultural structures, outside air replaces indoor air at a much higher air exchange rate, making it difficult to treat the incoming air using conventional commercial air purification systems.

An anti-viral and anti-bacterial agent material may be produced from dolomite, a natural mineral comprising calcium magnesium carbonate (Ca·Mg(CO3)2). Dolomite may be “activated” by producing a powder comprising calcined and partially hydrated dolomite which have anti-viral properties. For example, the use of dolomite in filter materials, such as masks, is described in Japanese Patent No. 4,621,590 to Mochigase Electrical Equipment, and in related International Patent Cooperation Treaty (PCT) Patent Application Publication No. WO2005013695, filed Aug. 10, 2004, the entire contents of which are incorporated herein by reference. In embodiments, the calcined and partially hydrated dolomite generates hydroxyl radicals which inactivate viruses by binding to the HA protein and envelope of the target virus.

Hydroxyls generated from calcined and partially hydrated dolomite may inactivate one or more pathogens, such as the H5N1 virus, a highly pathogenic avian influenza virus, found within barns and other similar structures in the agricultural industry.

### SUMMARY

An aspect relates to an air purification system for use in a structure, the system comprising: a housing configured for airflow therethrough; at least one anti-viral unit comprising a substrate material, and an anti-viral agent material supported on the substrate material.

In embodiments, the anti-viral agent material comprises a mineral powder containing a metal oxide and a metal hydroxide suitable for hydroxyl generation.

In embodiments, the anti-viral agent material comprises a mineral powder containing calcined and partially hydrated dolomite.

In embodiments, the farm air purification system further comprises one or more light sources, such as one or more UV light sources.

In embodiments, the at least one anti-viral unit further comprises activated charcoal.

In embodiments, the at least one anti-viral unit further comprises titanium dioxide.

In embodiments, the at least one anti-viral unit further comprises a blend of titanium dioxide and activated charcoal for hydroxyl generation in low humidity environments.

In embodiments, air passes through the farm air purification system to emit hydroxyls into the structure.

In embodiments, the hydroxyls neutralize viruses, odors, bacteria, and other pathogens in the structure.

Other aspects and features of the present disclosure will become apparent to those ordinarily skilled in the conventional art, upon review of the following description of the specific embodiments of the disclosure.

### BRIEF DESCRIPTION

Some of the embodiments will be described in detail, with references to the following Figures, wherein like designations denote like members, wherein:

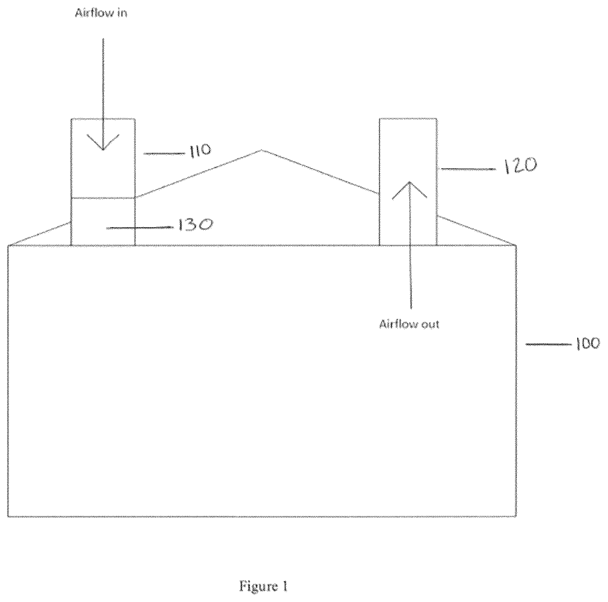

FIG. 1 is a schematic of a barn with the farm air purification system situated in line with an air intake; and

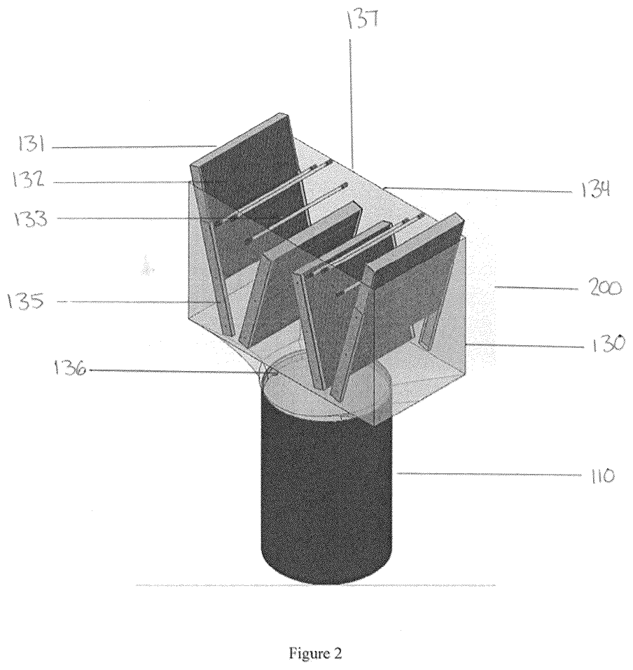

FIG. 2 is a perspective view of an air purification system according to embodiments of the invention.

### DETAILED DESCRIPTION

Generally, the present disclosure provides an air purification system. In some embodiments, the air purification system disclosed herein may be installed in an agricultural structure (such as a barn, for example). The air purification system may at least partially treat air flow within the agricultural structure. At least partially treating the airflow may include reducing or minimize bacterial and/or viral loads in the air. Embodiments disclosed herein may be more cost-effective for use in agricultural settings than conventional air purification systems, which may thereby increase farm revenue. Embodiments disclosed herein may also reduce barn odors, without sacrificing air exchange rates or requiring expensive upgrades to ventilation systems, such as installing HEPA filters or high-pressure fan systems.

In this disclosure the term “activated dolomite” is used herein to refer to the anti-viral agent comprising calcined and partially hydrated dolomite.

In embodiments, the system comprises at least one anti-viral unit supporting activated dolomite situated in line with the at least one air intake of an agricultural structure to promote hydroxyls into the air stream of the agricultural structure.

FIG. 1 shows an agricultural structure 100, which includes at least one air inlet 110 and at least one air outlet 120. While FIG. 1 shows one air intake 110 and one air outlet, the agricultural structure may include any number of air intakes and outlets, and embodiments are not limited to a particular number. Air flows into the agricultural structure 100 via the air intake 110 and air flows out of the agricultural structure 100 via the air outlet 120. Any suitable means of driving the airflow through the air intake 120 and the air outlet 120 may be used. For example, the air outlet 120 shown may include an air mover, such as a fan, to push air out through the outlet 120, thereby creating a negative pressure differential to draw air in through the air intake 110. The air intake 110 may be a chimney style air intake, as depicted in FIG. 1, or in another embodiment the air intake may be situated on the roof or the exterior walls of the agricultural structure. The air outlet 120 may be situated on the roof of the agricultural structure 100, or in another embodiment, the air outlet may be situated on the exterior walls of the agricultural structure.

FIG. 1 also shows an example air purification system 130, according to some embodiments. The air purification system 130 is installed inline with airflow in the agricultural structure 100. More particularly, the air purification system 130 in this embodiment is installed inline with the air intake 110, such that air flowing (from the outside environment) into the air intake 110 flows through the air purification system 130 as it enters the agricultural structure 100.

The air purification system 130 includes a housing configured for flowing air therethrough. The housing contains one or more anti-viral units, each comprising an antiviral material that emits hydroxyls into the airflow through embodiments of the system 130. Air flowing out of the air purification system 130 and into the agricultural structure 100 may thereby be infused with hydroxyls. The hydroxyls may, in turn, treat air within the agricultural structure by contacting and inactivating viruses or other pathogens.

The present disclosure is not limited to agricultural structures, or to using negative pressure ventilation. Embodiment of the air purification system may be configured for use with other types of ventilation systems whereby an air stream enters a building or other structure through an air intake.

FIG. 2 is a perspective view of the example air purification system 130, but embodiments are not limited to this example configuration. The farm air purification system 200 is situated in line with the air intake 110, such that the air stream passing through the air intake 110 passes through the farm air purification system 130. More specifically, in this example, the air purification system 130 includes a housing 134 having an air inlet end 137 and an air outlet end 136. The air inlet end 137 is coupled to the air intake 110. Airflow through the air purification system 130 from the intake 110 exits through the air outlet end 136 and continues through the agricultural structure 100 of FIG. 1.

In the air purification system 130 depicted in FIG. 2, each antiviral unit 131 is in the form of a filter panel 131 arranged within the housing 134. Embodiments are not limited to the filter panels. Any panel or other structure providing surface area suitable for supporting the anti-viral agent material may be used. Embodiments are also not limited to the number of filters 131 shown in FIG. 2. Fewer (e.g., a single filter) or more filters may be used in other embodiments depending on various factors, such as dimensions, materials, or other design considerations. The filters 131 are placed at an angle, relative to the path from the air inlet end 137 to the air outlet end 136, as depicted in FIG. 2.

The filter 131 comprises a substrate material 132 supporting an anti-viral agent material. The substrate material may, for example, be an air permeable filter material such as woven fabric or non-woven fabric, although embodiments are not limited to these types of materials. Methods for supporting the anti-viral agent material onto the substrate material 132 include mixing the anti-viral agent material, an adhesive, water, and a dispersant to prepare an aqueous solution containing the partially dissolved anti-viral agent material, dipping the substrate material into the prepared aqueous solution, and subsequently drying the substrate material. PCT Patent Application Publication No. WO/2005/013695, filed Aug. 10, 2004, discloses an example process that may be used to prepare the anti-viral agent material and methods used to prepare the material supporting the anti-viral agent material. However, embodiments are not limited to the particular filter materials and/or anti-viral agent material(s) disclosed in PCT Patent Application Publication No. WO2005013695.

The anti-viral agent material may comprise a powder of calcined and partially hydrated dolomite. The powder of calcined and partially hydrated dolomite may be prepared by the processes of calcination and hydration (slaking) of a raw dolomite mineral (Ca·Mg(CO₃)₂) under special operational conditions to produce a mixture of a metal oxide (CaO and MgO) and hydroxide (Mg(OH)₂ and Ca(OH)₂) that cause a reaction generating hydroxyl radicals.

During the calcination process of raw dolomite, Ca·Mg(CO₃)₂ may decompose at a temperature between 750° C. and 800° C. into the following products:

Ca·Mg(CO₃)₂→CaCO₃+MgO+CO₂

As the temperature increases, CaCO₃ may decompose at temperatures between 900° C. and 1000° C. into the following products:

CaCO₃+MgO→CaO+MgO+CO₂

Following calcination, a material primarily composed of calcium oxide (CaO) and magnesium oxide (MgO) may be produced. While the calcined dolomite is still at high temperatures, water may be poured over the calcined dolomite to partially hydrate the dolomite. The resulting product may contain both metal oxides and hydroxides and may be pulverized into a mineral powder, with an average particle size of 0.1 μm to 60 μm.

Hydroxyl radicals may be generated from the reaction between the metal oxides and hydroxides which coexist in the mineral powder. PCT Patent Application Publication No. WO/2009/098908, the entire contents of which are incorporated by reference, discloses the method in which the metal oxide and hydroxide enable the generation of hydroxyl radicals. Embodiments are not limited to the powder of a metal oxide and hydroxide resulting from the calcination and slaking of raw dolomite. For example, a powder of an oxide of one or more metals selected from the group consisting of alkali metals, alkaline earth metals, metals of groups 4 to 12 of the periodic table, or aluminum with one or more hydroxides selected from alkali metal hydroxides, alkaline earth metal hydroxides, iron hydroxide, copper hydroxide, zinc hydroxide, aluminum hydroxide, or ammonium hydroxide may be used.

The hydroxyls that are generated from the powder of calcined and partially hydrated dolomite that is supported on the substrate material 132 may be emitted into the agricultural structure with the air stream that passes through the farm air purification system. Hydroxyls may independently inactivate viruses, VOCs, odors, and other pathogens including, but not limited to, highly pathogenic avian influenza viruses (H5N1), Staphylococcus aureus, MRSA, Enterococcus, vancomycin-resistant Enterococcus, Colon bacillus, Salmonella, Pseudomonas aeruginosa, multi-drug resistant Pseudomonas aeruginosa, and Bacillus subtilis.

In embodiments, the anti-viral units 131 of the farm air purification system 130 may also incorporate activated charcoal with the antiviral agent material. For example, a combination of activated charcoal and powder of calcined and partially hydrated dolomite may be supported on the substrate material, which may enhance hydroxyl radical generation into the air stream.

In embodiments, the anti-viral units 131 of the farm air purification system 130 may also incorporate titanium dioxide (TiO₂) with the antiviral agent material, which may permit stable hydroxyl radical generation in low-humidity environments. For example, the substrate material of the anti-viral units 131 may support a mixture of activated dolomite and titanium dioxide. In another example, the substrate material may support a mixture of activated dolomite, titanium dioxide, and activated carbon.

In embodiments, the farm air purification system 130 may further comprise one or more light sources. In this example, the light sources are ultraviolet (UV) light sources 133, but embodiments are not limited to UV light sources. FIG. 2 shows a plurality of example UV light sources 133 shown in FIG. 2 positioned between adjacent anti-viral units 131. The UV light sources output UV light which is incident on the antiviral agent material of the anti viral units 131 with the powder of calcined and partially hydrated dolomite to increase hydroxyl radical generation into the air stream.

In embodiments, the farm air purification system 130 may comprise one or more UV light sources 133, activated charcoal, and the powder of calcined and partially hydrated dolomite to increase hydroxyl radical generation into the air stream.

With reference again to FIG. 2, the housing 134 of the farm air purification system 130 may be rectangular box shaped. However, embodiments are not limited to rectangular shaped housing. For example, the housing of the farm air purification system may be circular or square shaped, depending on the shape of the air intake.

The anti-viral unit 131 may be attached to a moveable support 135 to adjust the height and angle of the unit within the farm air purification system 130. The anti-viral unit 131 may be placed in a position that does not significantly obstruct airflow, while facilitating the production of hydroxyl radicals in the air stream.

Although the present invention has been disclosed in the form of embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module.

1. An air purification system for use in a structure, the system comprising:
a housing configured for airflow therethrough;
at least one anti-viral unit comprising a substrate material and an anti-viral agent material supported on the substrate material.

2. The system of claim 1, wherein the anti-viral agent material comprises a mineral powder containing a metal oxide and a metal hydroxide suitable for hydroxyl generation.

3. The system of claim 1, wherein the anti-viral agent material comprises a mineral powder containing calcined and partially hydrated dolomite.

4. The system of claim 1, where the farm air purification system further comprises one or more light sources.

5. The system of claim 4, wherein the one or more light sources comprise one or more UV light sources.

6. The system of claim 1, where the at least one anti-viral unit further comprises activated charcoal.

7. The system of claim 1, where the at least one anti-viral unit further comprises titanium dioxide.

8. The system of claim 1, where the at least one anti-viral unit further comprises a blend of titanium dioxide and activated charcoal for hydroxyl generation in low humidity environments.

9. The system of claim 1, where air passes through the farm air purification system to emit hydroxyls into the structure.

10. The system of claim 1, where the hydroxyls neutralize viruses, odors, bacteria, and other pathogens in the structure.
